(19) United States
(12) Patent Application Publication
(10) Pub. No.: US 2026/0065482 A1
(43) Pub. Date: Mar. 5, 2026

(54) MEDICAL INFORMATION PROCESSING APPARATUS, STORAGE MEDIUM, AND METHOD

(71) Applicants: The Johns Hopkins University, Baltimore, MD (US); CANON MEDICAL SYSTEMS CORPORATION, Tochigi (JP)

(72) Inventors: Kristin REDMOND, Severna Park, MD (US); Junghoon LEE, Woodstock, MD (US); Chetan BETTEGOWDA, Lutherville, MD (US); Asateru KIMURA, Otawara (JP); Kazumasa NORO, Shioya (JP)

(73) Assignees: The Johns Hopkins University, Baltimore, MD (US); CANON MEDICAL SYSTEMS CORPORATION, Tochigi (JP)

(21) Appl. No.: 18/817,364

(22) Filed: Aug. 28, 2024

(51) Int. Cl. G06T 7/00; G06V 20/69; G16B 20/20; G16H 50/20

(52) U.S. Cl. CPC G06T 7/0016; G06V 20/698; G16B 20/20; G16H 50/20; G06T 2207/30024; G06T 2207/30096; G06V 2201/03

(57) ABSTRACT

A medical information processing apparatus according to an embodiment includes processing circuitry. The processing circuitry is configured to obtain data related to circulating tumor deoxyribonucleic acid (ctDNA) of a subject. The processing circuitry is configured to specify a gene on the basis of information about a false negative in a result of estimating a genetic mutation in a tumor tissue of the subject based on the data related to the ctDNA. With respect to the gene specified on the basis of the information about the false negative, the processing circuitry is configured to calculate a result of estimating the genetic mutation in the tumor tissue, on the basis of a medical image of the tumor tissue of the subject.

### FIELD

Embodiments described herein relate generally to a medical information processing apparatus, a storage medium, and a method.

### BACKGROUND

Known methods for judging effects of cancer treatment include a method by which genetic mutations in a tumor are monitored. For example, by regularly checking how genetic mutations are in a tumor tissue, it is possible to judge whether or not the treatment currently performed has an effect. In this situation, among types of cell free deoxyribonucleic acid (cell free DNA), because DNA originating from tumors called circulating tumor DNA (ctDNA) is a specimen that can be obtained in a non-invasive and convenient manner, it is expected that ctDNA can be applied to monitoring genetic mutations; however, because ctDNA has a small sample quantity and a short half-life period, analyses using ctDNA have a high false-negative rate. Thus, there are situations where genetic mutations in a tumor may not accurately be grasped and the genetic mutations therefore may not be accurately monitored.

### BRIEF DESCRIPTION OF THE DRAWINGS

FIG. 1 is a diagram illustrating an exemplary configuration of a medical information processing apparatus according to a first embodiment;

FIG. 2 is a drawing for explaining matching degrees with genetic mutations in a tumor tissue specimen;

FIG. 3 is a drawing for explaining an outline of a process performed by the medical information processing apparatus according to the first embodiment;

FIG. 4 is a flowchart illustrating a processing procedure of processes performed by processing functions included in processing circuitry of the medical information processing apparatus according to the first embodiment;

FIG. 5 is a drawing for explaining an example of a process performed by the medical information processing apparatus according to the first embodiment;

FIG. 6 is a drawing for explaining an example of correspondence information according to the first embodiment;

FIG. 7 is a drawing illustrating an example of display information according to the first embodiment; and

FIG. 8 is a drawing for explaining an example of a process performed by a judging function according to the first embodiment.

### DETAILED DESCRIPTION

A medical information processing apparatus according to an embodiment includes processing circuitry. The processing circuitry is configured to obtain data related to circulating tumor deoxyribonucleic acid (ctDNA) of a subject. The processing circuitry is configured to specify a gene on the basis of information about a false negative in a result of estimating a genetic mutation in a tumor tissue of the subject based on the data related to the ctDNA. With respect to the gene specified on the basis of the information about the false negative, the processing circuitry is configured to calculate a result of estimating the genetic mutation in the tumor tissue, on the basis of a medical image of the tumor tissue of the subject.

Exemplary embodiments of a medical information processing apparatus, a storage medium, and a method will be explained in detail below, with reference to the accompanying drawings. The medical information processing apparatus, the storage medium, and the method of the present disclosure are not limited by the embodiments described below.

### First Embodiment

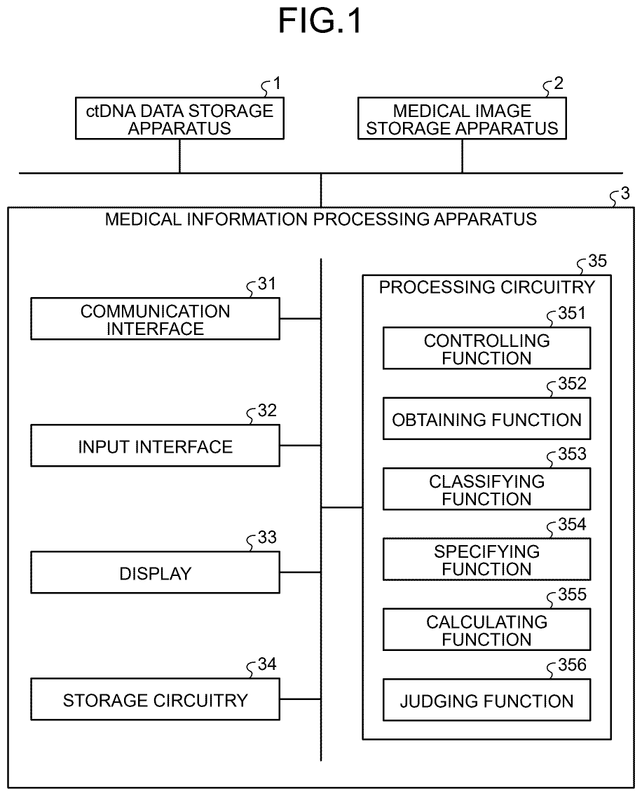

FIG. 1 is a diagram illustrating an exemplary configuration of a medical information processing apparatus according to a first embodiment. For example, as illustrated in FIG. 1, a medical information processing apparatus 3 according to the present embodiment is communicably connected to a ctDNA data storage apparatus 1 and a medical image storage apparatus 2 via a network. To the network illustrated in FIG. 1, other various types of apparatuses and systems may be connected.

The ctDNA data storage apparatus 1 is configured to store therein data (ctDNA data) related to circulating tumor deoxyribonucleic acid (ctDNA) of a subject. More specifically, the ctDNA data storage apparatus 1 is configured to store therein information about genetic mutations in the ctDNA of a cancer patient (the subject) who underwent a liquid biopsy test. For example, the ctDNA data storage apparatus 1 is configured to store therein the information about the genetic mutations in the ctDNA included in DNA segments called cell free DNA (cfDNA) circulating in the plasma isolated from blood of the cancer patient.

For example, by analyzing the cfDNA included in the plasma of the cancer patient through a next-generation sequencing (NGS) scheme and a digital polymerase chain reaction (dPCR) scheme, it is possible to identify the genetic mutations in the ctDNA and to calculate mutant allele frequency (MAF) of each gene. MAF is calculated by dividing the quantity of variants in PCR-amplified genes by a total number of PCR-amplified genes and is a value that varies in correspondence with the quantity of observed gene mutations (i.e., a result of quantifying the degree by which the gene mutations are detected). When there is no gene mutation, MAF is 0.

Blood is collected from the cancer patient before and after treatment, during a follow-up period, or the like, so as to obtain the abovementioned information about the gene mutations in the ctDNA. The ctDNA data storage apparatus 1 is configured to store therein the information about the gene mutations in the ctDNA for each cancer patient. For example, the ctDNA data storage apparatus 1 may be realized by using a computer machine such as a server or a workstation.

The medical image storage apparatus 2 is configured to store therein various types of medical images related to the subject. More specifically, the medical image storage apparatus 2 is configured to store therein a medical image rendering a tumor tissue of the cancer patient (the subject). For example, the medical image storage apparatus 2 is configured to store therein, with respect to each cancer patient, a medical image acquired at the time when the liquid biopsy test is performed. For example, the medical images stored by the medical image storage apparatus 2 may be acquired by an X-ray diagnosis apparatus, an X-ray Computed Tomography (CT) apparatus, a Magnetic Resonance Imaging (MRI) apparatus, an ultrasound diagnosis apparatus, a Single Photon Emission Computed Tomography (SPECT) apparatus, a Positron Emission computed Tomography (PET) apparatus, and/or the like.

For example, the medical image storage apparatus 2 may be realized by using a computer machine such as a server or a workstation. Further, for example, the medical image storage apparatus 2 may be realized by using a Picture Archiving and Communication System (PACS) or the like and configured to store the medical images therein in a format compliant with a Digital Imaging and Communications in Medicine (DICOM) scheme.

The medical information processing apparatus 3 is configured to perform various types of processes by using medical information of the subject. More specifically, the medical information processing apparatus 3 is configured to receive the medical information from the ctDNA data storage apparatus 1 and the medical image storage apparatus 2 via the network and to perform the various types of processes by using the medical information. For example, the medical information processing apparatus 3 may be realized by using a computer machine such as a server or a workstation.

For example, the medical information processing apparatus 3 includes a communication interface 31, an input interface 32, a display 33, storage circuitry 34, and processing circuitry 35.

The communication interface 31 is configured to control transfer and communication of various types of data transmitted and received between the medical information processing apparatus 3 and other apparatuses connected via the network. More specifically, the communication interface 31 is connected to the processing circuitry 35 and is configured to transmit data received from any of the other apparatuses to the processing circuitry 35 and to transmit data received from the processing circuitry 35 to any of the other apparatuses. For example, the communication interface 31 may be realized by using a network card, a network adaptor, and a Network Interface Controller (NIC), or the like.

The input interface 32 is configured to receive operations to input various types of instructions and various types of information from a user. More specifically, the input interface 32 is connected to the processing circuitry 35 and is configured to convert the input operations received from the user into electrical signals and to output the electrical signals to the processing circuitry 35. For example, the input interface 32 may be realized by using a trackball, a switch button, a mouse, a keyboard, a touchpad on which input operations can be performed by touching an operation surface thereof, a touch screen in which a display screen and a touchpad are integrally formed, a contactless input interface using an optical sensor, an audio input interface, and/or the like. Further, in the present disclosure, the input interface 32 does not necessarily need to include physical operation component parts such as the mouse, the keyboard, and/or the like. For instance, possible examples of the input interface 32 include electrical signal processing circuitry configured to receive an electrical signal corresponding to an input operation from an external input mechanism provided separately from the apparatus and to output the electrical signal to controlling circuitry.

The display 33 is configured to display various types of information and various types of data. More specifically, the display 33 is connected to the processing circuitry 35 and is configured to display various types of information and various types of data received from the processing circuitry 35. For example, the display 33 may be realized by using a liquid crystal display, a Cathode Ray Tube (CRT) display, a touch panel, LED display, or the like.

The storage circuitry 34 is configured to store therein various types of data and various types of programs. More specifically, the storage circuitry 34 is connected to the processing circuitry 35 and is configured to store therein data received from the processing circuitry 35 and to read and transmit any of the data stored therein to the processing circuitry 35. Further, the storage circuitry 34 is configured to store therein correspondence information and a prediction model to be used by the processing circuitry 35. The correspondence information and the prediction model will be explained in detail later. For example, the storage circuitry 34 may be realized by using a semiconductor memory element such as a Random Access Memory (RAM) or a flash memory, or a hard disk, an optical disk, or the like.

The processing circuitry 35 is configured to control the entirety of the medical information processing apparatus 3. For example, the processing circuitry 35 is configured to perform various types of processes, in accordance with the input operations received from the user via the input interface 32. For example, the processing circuitry 35 is configured to receive data transmitted by any of the other apparatuses via the communication interface 31 and to store the received data into the storage circuitry 34. Further, for example, the processing circuitry 35 is configured to transmit any of the data received from the storage circuitry 34 to the communication interface 31, so as to transmit the data to any of the other apparatuses. Further, for example, the processing circuitry 35 is configured to cause the display 33 to display any of the data received from the storage circuitry 34.

The exemplary configuration of the medical information processing apparatus 3 according to the present embodiment has thus been explained. The medical information processing apparatus 3 according to the present embodiment is installed in a medical facility such as a hospital or a clinic and is configured to assist various types of diagnosing processes and treatment plan making processes performed by the user such as a medical doctor. More specifically, the medical information processing apparatus 3 is configured to enhance the precision level for estimating genetic mutations in a tumor tissue, by combining the ctDNA data of the subject (the cancer patient) with image feature values of the tumor tissue of the subject.

As explained above, because ctDNA is a specimen that can be obtained in a non-invasive and convenient manner, it is expected that ctDNA can be applied to monitoring genetic mutations in tumor tissues; however, because the sample quantity is small and the half-life period is short, there may be situations where, depending on the timing of liquid biopsy tests, the result may be a false negative where no ctDNA is detected. Meanwhile, there is a certain correlation between image feature values of a tumor tissue in a medical image and genetic mutations in the tumor tissue. It is therefore possible to estimate genetic mutations in a tumor tissue on the basis of image feature values.

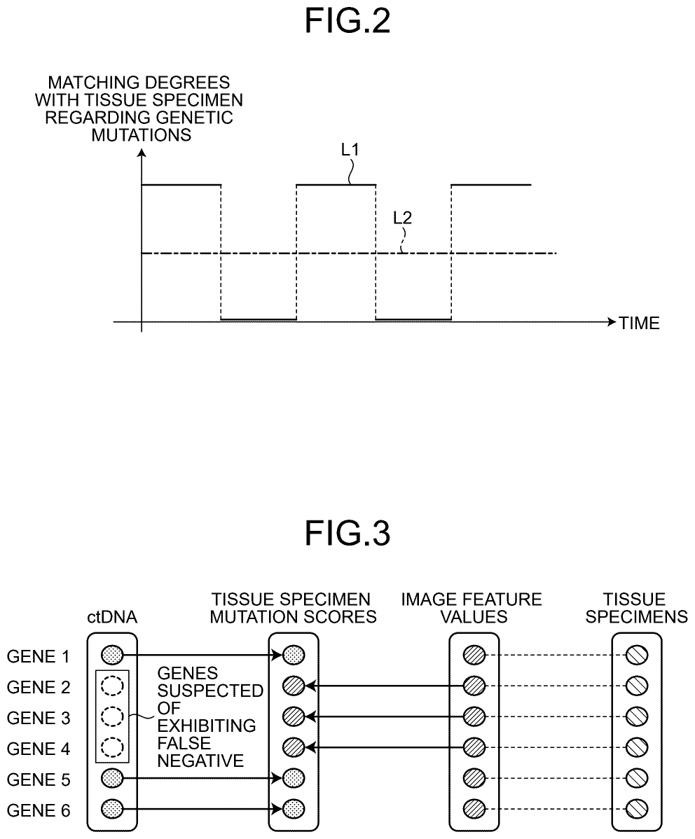

FIG. 2 is a drawing for explaining degrees of matching (hereinafter, “matching degrees”) with genetic mutations in a tumor tissue specimen. In FIG. 2, the vertical axis expresses the matching degrees with the genetic mutations, whereas the horizontal axis expresses timing of analyses (time). Further, in FIG. 2, the line L1 indicates matching degrees based on ctDNA, whereas the line L2 indicates matching degrees based on image feature values. In other words, the line L1 indicates the matching degrees between genetic mutations in the tumor identified while using ctDNA and genetic mutations in the tumor identified while using DNA extracted from the tumor tissue. Further, the line L2 indicates the matching degrees between genetic mutations identified from image feature values of the tumor tissue and genetic mutations in the tumor identified while using the DNA extracted from the tumor tissue.

As indicated by the line L1 in FIG. 2, the genetic mutations identified from the ctDNA exhibit a higher matching degree with the genetic mutations identified from the DNA of the tumor tissue, as compared with the example using the image feature values; however, depending on the timing of the analysis, there is hardly any matching in some situations. In other words, when the plasma contains no ctDNA, the result may be a false negative indicating that there are no genetic mutations, even though the tumor tissue has genetic mutations.

In contrast, as indicated by the line L2 in FIG. 2, the genetic mutations identified from the image feature values exhibit a lower matching degree with the genetic mutations identified from the DNA of the tumor tissue, as compared with the example using the ctDNA, and the matching degree is more constant.

Thus, the medical information processing apparatus 3 according to the present embodiment is configured to enhance the level of precision for estimating genetic mutations in a tumor tissue, by using image feature values for estimating genetic mutations that failed to be detected with ctDNA. FIG. 3 is a drawing for explaining an outline of a process performed by the medical information processing apparatus 3 according to the first embodiment. As illustrated in FIG. 3, for example, the medical information processing apparatus 3 is configured to obtain information about genetic mutations identified from ctDNA, with respect to genes 1 to 6. In this situation, when the obtained information includes genes (genes 2, 3, and 4) suspected of exhibiting a false negative, the medical information processing apparatus 3 is configured, with respect to genes 2, 3, and 4, to obtain image feature values that are in correlation with genetic mutations of the genes identified from a tissue specimen and to further calculate tissue specimen mutation score for evaluating the genetic mutations in the tumor tissue by using the obtained image feature values. In contrast, with respect to genes 1, 5, and 6 in which genetic mutations were identified from ctDNA, the medical information processing apparatus 3 is configured to calculate tissue specimen mutation scores by using the information about the genetic mutations identified from the ctDNA.

In the present embodiment, as illustrated in FIG. 1, for example, the processing circuitry 35 of the medical information processing apparatus 3 is configured to perform various types of processes including the process explained with reference to FIG. 3, by executing a controlling function 351, an obtaining function 352, a classifying function 353, a specifying function 354, a calculating function 355, and a judging function 356. In this situation, the processing circuitry 35 is an example of processing circuitry.

In accordance with operations received via the input interface 32, the controlling function 351 is configured to generate various types of Graphical User Interfaces (GUIs) and various types of display information and to exercise control so that the display 33 displays the generated GUIs and information. For example, the controlling function 351 is configured to cause the display 33 to display results of the processes performed by the functions. Further, the controlling function 351 is also capable of generating and displaying various types of display images on the basis of medical images obtained by the obtaining function 352.

The obtaining function 352 is configured to obtain, via the communication interface 31, medical information of the subject from the ctDNA data storage apparatus 1 and the medical image storage apparatus 2 and to store the obtained medical information into the storage circuitry 34. More specifically, the obtaining function 352 is configured to obtain data (ctDNA data) related to the ctDNA of the subject, from the ctDNA data storage apparatus 1. Also, the obtaining function 352 is configured to obtain the medical image rendering the tumor tissue of the subject from the medical image storage apparatus 2.

The classifying function 353 is configured to classify genes into gene groups. Processes performed by the classifying function 353 will be explained in detail later.

The specifying function 354 is configured to specify genes on the basis of information about false negatives in results of estimating genetic mutations in the tumor tissue of the subject based on the data related to the ctDNA. More specifically, the specifying function 354 is configured to specify a first gene having a high possibility of exhibiting a false negative in the results of estimating the genetic mutations based on the data related to the ctDNA. For example, on the basis of the MAF value of each of the genes, the specifying function 354 is configured to judge the possibility of exhibiting a false negative in the results of estimating the genetic mutations. In an example, the specifying function 354 is configured to specify a gene having a smaller MAF value than a threshold value, as the first gene having the high possibility of exhibiting a false negative. Processes performed by the specifying function 354 will be explained in detail later.

With respect to the gene specified on the basis of the information about the false negatives, the calculating function 355 is configured to calculate a result of estimating the genetic mutations in the tumor tissue, on the basis of the medical image of the tumor tissue of the subject. More specifically, the calculating function 355 is configured to calculate, with respect to the first gene, a mutation score (a tissue specimen mutation score) for estimating the genetic mutations on the basis of the medical image of the tumor tissue and to calculate, with respect to a second gene other than the first gene, a mutation score (a tissue specimen mutation score) for estimating the genetic mutations on the basis of the data related to the ctDNA. For example, as the result of estimating mutations of the gene specified on the basis of the information about false negatives, the calculating function 355 is configured to calculate the image feature values in the medical image of the tumor tissue. Processes performed by the calculating function 355 will be explained in detail later.

The judging function 356 is configured to judge an effect of treatment on the tumor tissue, on the basis of results of estimating the genetic mutations in the tumor tissue before and after the treatment performed on the tumor tissue of the subject. Processes performed by the judging function 356 will be explained in detail later.

The processing circuitry 35 described above may be realized by using one or more processors, for example. In that situation, the processing functions described above are stored in the storage circuitry 34 in the form of computer-executable programs. Further, the processing circuitry 35 is configured to realize the functions corresponding to the programs, by reading and executing the programs stored in the storage circuitry 34. In other words, the processing circuitry 35 that has read the programs has the processing functions illustrated in FIG. 1.

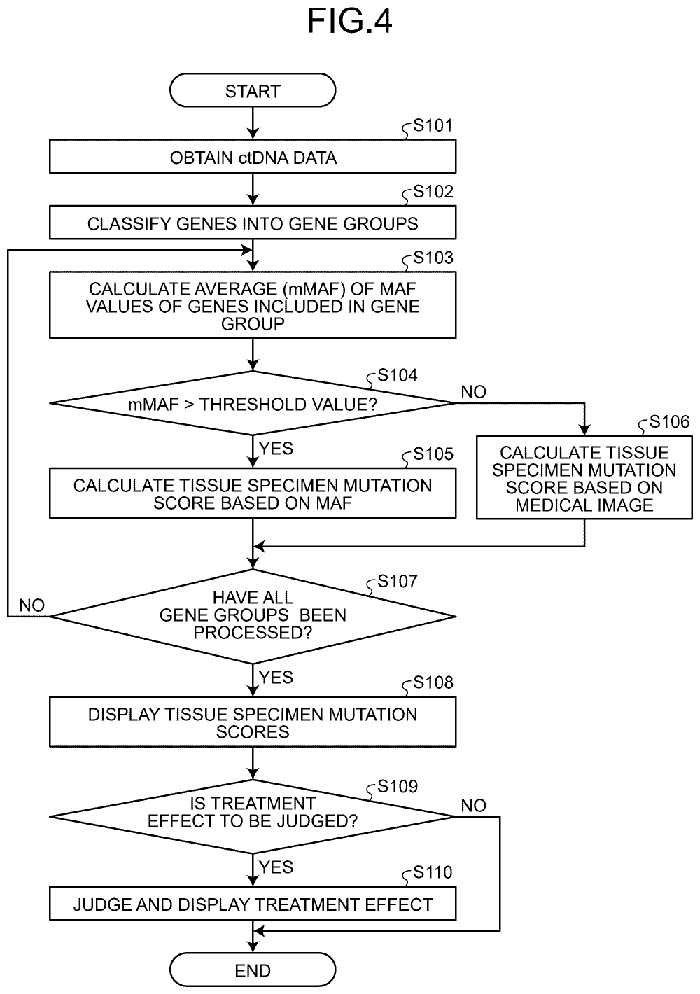

Next, a procedure of processes performed by the medical information processing apparatus 3 will be explained with reference to FIG. 4, and after that, details of the processes will be explained. FIG. 4 is a flowchart illustrating a processing procedure of processes performed by the processing functions included in the processing circuitry 35 of the medical information processing apparatus 3 according to the first embodiment. In this situation, the medical information processing apparatus 3 according to the present embodiment is able to calculate the tissue specimen mutation score with respect to each of the genes, as explained with reference to FIG. 3. Alternatively, the medical information processing apparatus 3 is also able to classify the plurality of genes into gene groups and to further calculate a tissue specimen mutation score with respect to each of the gene groups. In other words, the specifying function 354 may be configured to specify a gene group having a high possibility of exhibiting a false negative in an estimation result regarding the genetic mutations estimated with respect to each of the gene groups. With respect to the specified gene group, the calculating function 355 may be configured, on the basis of the medical image of the tumor tissue of the subject, to calculate a result of estimating the genetic mutations in the tumor tissue. In the following sections, the example will be explained in which the tissue specimen mutation score is calculated with respect to each of the gene group.

For example, as illustrated in FIG. 4, in the present embodiment, the obtaining function 352 obtains the ctDNA data of the subject (the cancer patient) from the ctDNA data storage apparatus 1 (step S101). For example, in response to a ctDNA obtaining operation performed via the input interface 32, the obtaining function 352 obtains the ctDNA data of the cancer patient to be analyzed. This process is realized, for example, as a result of the processing circuitry 35 invoking and executing a program corresponding to the obtaining function 352 from the storage circuitry 34.

Subsequently, the classifying function 353 classifies the genes into gene groups (step S102). This process is realized, for example, as a result of the processing circuitry 35 invoking and executing a program corresponding to the classifying function 353 from the storage circuitry 34.

After that, the specifying function 354 calculates an average (mMAF) of the MAF values of the genes included in each of the gene groups on the basis of the ctDNA data (step S103) and judges whether or not the calculated mMAF exceeds a threshold value (step S104). This process is realized, for example, as a result of the processing circuitry 35 invoking and executing a program corresponding to the specifying function 354 from the storage circuitry 34.

In this situation, when the mMAF exceeds the threshold value (step S104: Yes), the calculating function 355 calculates a tissue specimen mutation score based on the MAF (step S105). On the contrary, when the mMAF is equal to or smaller than the threshold value (step S104: No), the calculating function 355 calculates a tissue specimen mutation score based on the medical image (step S106). These processes are realized, for example, as a result of the processing circuitry 35 invoking and executing a program corresponding to the calculating function 355 from the storage circuitry 34.

Subsequently, the specifying function 354 judges whether or not all the gene groups have been processed (step S107). When all the gene groups have not been processed (step S107: No), the specifying function 354 returns to step S103 and performs the process. On the contrary, when all the gene groups have been processed (step S107: Yes), the controlling function 351 exercises control so that the tissue specimen mutation scores are displayed (step S108). These processes are realized, for example, as a result of the processing circuitry 35 invoking and executing the programs corresponding to the specifying function 354 and the controlling function 351 from the storage circuitry 34.

After that, the judging function 356 determines whether or not a treatment effect is to be judged (step S109). In this situation, when the treatment effect is to be judged (step S109: Yes), the judging function 356 judges the treatment effect on the basis of tissue specimen mutation scores calculated at mutually-different points in time, so that the controlling function 351 exercises control to cause a judgement result to be displayed (step S110). On the contrary, when the treatment effect is not to be judged (step S109: No), the medical information processing apparatus 3 ends the process.

Next, details of the processes performed by the medical information processing apparatus 3 will be explained.

The ctDNA Data Obtaining Process

As explained at step S101 in FIG. 4, the obtaining function 352 is configured to obtain, from the ctDNA data storage apparatus 1, the ctDNA data of the subject subject to the analysis, in response to the ctDNA obtaining operation performed via the input interface 32. For example, with respect to the designated cancer patient, the obtaining function 352 is configured to obtain the results regarding the MAF values of the genes obtained from the liquid biopsy test. In addition, the obtaining function 352 may also obtain the medical image of the designated cancer patient at this time. For example, the obtaining function 352 is configured to obtain, from the medical image storage apparatus 2, a medical image acquired substantially at the same time as (or at a time close to) when the obtained ctDNA data was acquired (when the liquid biopsy test was performed).

In the present example, the operation in the obtaining process at step S101 may be started according to the user's instruction received via the input interface 32 as described above or may be started automatically. In the latter situation, for example, the obtaining function 352 is configured to monitor the ctDNA data storage apparatus 1 so that ctDNA data is automatically obtained every time a new piece of ctDNA data is stored.

### The Gene Group Classifying Process

As explained at step S102 in FIG. 4, the classifying function 353 is configured to classify the genes into the gene groups. More specifically, the classifying function 353 is configured to classify the genes into the gene groups in accordance with functions of the genes. For example, the classifying function 353 is configured to classify the genes into gene groups such as a gene group related to cell growth, a gene group related to DNA repair, and a gene group related to cell nucleus formation.

In this situation, the rule for classifying the genes does not necessarily need to be based on the functions of the genes as described above. It is acceptable to use other arbitrary rules. For example, it is also acceptable to form a gene group by gathering a plurality of genes of which genetic mutations are observed only in a small number of subjects.

### The False Negative Gene Specifying Process

As explained at steps S103 and S104 in FIG. 4, the specifying function 354 is configured to specify the gene groups each having a high possibility of exhibiting a false negative in the result of estimating the genetic mutations, by calculating the mMAF of each gene group on the basis of the ctDNA data and judging whether or not the calculated mMAF values exceed the threshold value.

As mentioned above, the MAF is obtained by quantifying the degree by which genetic mutations are detected. When the result is negative, the MAF is close to 0. However, depending on the timing of the analysis, there is a possibility that, as for the genetic mutations identified from ctDNA, the MAF numerical values may exhibit a false negative where no genetic mutation is detected. To cope with this situation, the specifying function 354 specifies one or more gene groups of which the mMAF value does not exceed the threshold value, as the gene groups each having the possibility of exhibiting a false negative in the result of estimating the genetic mutations.

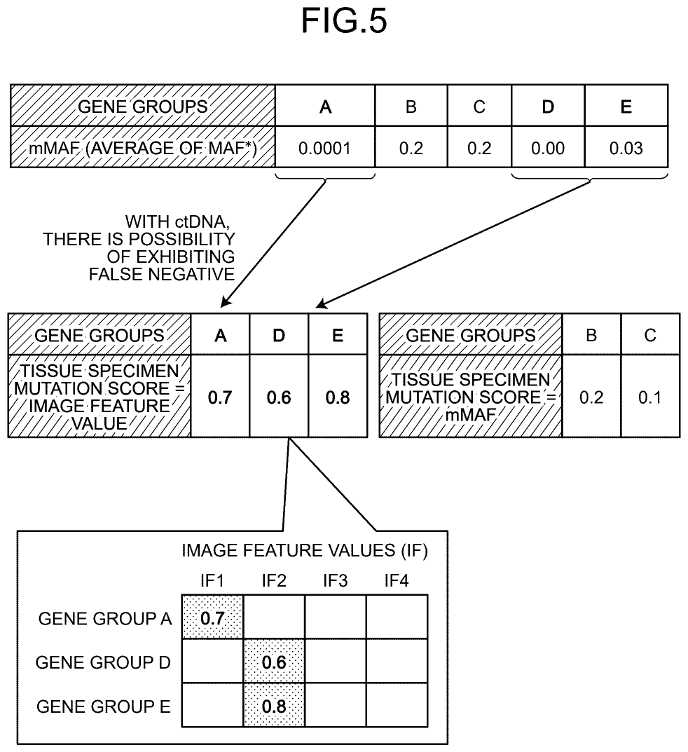

FIG. 5 is a drawing for explaining an example of a process performed by the medical information processing apparatus 3 according to the first embodiment. For example, as indicated in the top table in FIG. 5, with respect to gene group A, the specifying function 354 calculates an average value of the MAF values of the genes included in the group as “mMAF: 0.0001”. Similarly, the specifying function 354 calculates “mMAF: 0.2” for gene group B, “mMAF: 0.1” for gene group C, “mMAF: 0.00” for gene group D, and “mMAF: 0.03” for gene group E.

After that, the specifying function 354 is configured to specify one or more gene groups of which the mMAF is equal to or smaller than the threshold value, by comparing the calculated mMAF values with the threshold value. In this situation, for example, when the threshold value is set to “0.05”, for example, the specifying function 354 specifies gene groups A, D, and E satisfying “mMAF≤0.05” as the gene groups having the possibility of exhibiting a false negative. In this situation, the threshold value “0.05” presented above is merely an example, and the numerical value serving as the threshold value may be set as appropriate. For example, it is also acceptable to set a threshold value for each of different types of cancer or for each of different types of genes (or gene groups).

### The Tissue Specimen Mutation Score Calculating Process

As explained at steps S105 and S106 in FIG. 4, the calculating function 355 is configured to calculate the tissue specimen mutation scores based on the MAF with respect to the gene groups (the second gene) satisfying “mMAF>the threshold value” and to calculate the tissue specimen mutation scores based on the medical image with respect to the gene groups (the first gene) satisfying “mMAF≤the threshold value”. In other words, with respect to each of the gene groups satisfying “mMAF≤the threshold value”, the calculating function 355 is configured to calculate the tissue specimen mutation score on the basis of the image feature values of the tumor tissue.

For example, as illustrated in the middle table in FIG. 5, the calculating function 355 is configured to calculate the tissue specimen mutation scores from the image feature values with respect to gene groups A, D, and E specified as having the possibility of exhibiting a false negative on the basis of the MAF values of the ctDNA. In contrast, the calculating function 355 is configured to calculate the tissue specimen mutation scores from the mMAF with respect to gene groups B and C that were not specified as having a possibility of exhibiting a false negative.

In this situation, the correlation between the gene groups and the image feature values has been specified in advance, so that the correspondence information is stored in the storage circuitry 34. For example, with respect to each of the gene groups, an image feature value (e.g., a pixel value or a shape) corresponding to having a genetic mutation in a tumor tissue is compared with an image feature value corresponding to having no genetic mutation, so as to specify image feature values having significant differences. Further, the correspondence information keeping the specified image feature values in correspondence with the relevant gene groups is stored into the storage circuitry 34.

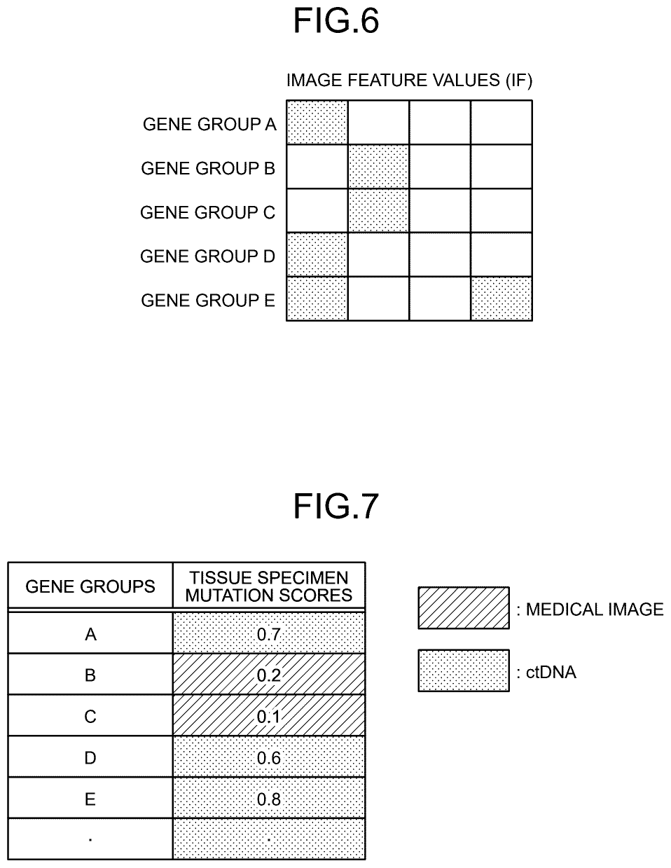

FIG. 6 is a drawing for explaining an example of the correspondence information according to the first embodiment. For example, as illustrated in FIG. 6, the storage circuitry 34 stores therein the correspondence information keeping each of gene groups A to E in correspondence with image feature values (“image feature (IF)”) that are in correlation with genetic mutations. In this situation, examples of the IF include morphological features (size, shapes, etc.) of tumors and features related to pixel values (a feature related to a frequency distribution, a feature related to a spatial distribution, etc.).

On the basis of the correspondence information stored in the storage circuitry 34, the calculating function 355 is configured to specify the image feature values that are in correlation with the gene groups specified as having the possibility of exhibiting a false negative. Further, the calculating function 355 is configured to calculate the specified image feature values from the medical image of the subject obtained by the obtaining function 352. For example, as illustrated in the bottom table in FIG. 5, the calculating function 355 is configured to specify an image feature value “IF1” that is in correlation with genetic mutations of gene group A on the basis of the correspondence information and to further calculate an image feature value “0.7” related to IF1 from the medical image of the subject.

Similarly, with respect to gene groups D and E, the calculating function 355 is configured to specify an image feature value “IF2” that is in correlation with genetic mutations and to further calculate image feature values “0.6” and “0.8” related to IF2 from the medical image of the subject. Although FIG. 5 illustrates the example in which the single image feature value is specified as the image feature value that is in correlation with genetic mutations, a plurality of image feature values may be specified as image feature values that are in correlation with genetic mutations, in actuality. In that situation, the calculating function 355 is able to calculate a tissue specimen mutation score by combining together the specified plurality of image feature values.

As explained above, depending on whether there is a possibility of exhibiting a false negative in the results of estimating the genetic mutations, the calculating function 355 is configured to switch the information used for calculating the tissue specimen mutation score. Because the information serving as the basis is switched in accordance with the situations in this manner, the calculating function 355 is configured to perform a standardization process or a normalization process on the image feature values and on the mMAF, to make it possible to compare the values with one another. For example, the calculating function 355 performs the standardization process or the normalization process on the values by using Expression (1) or (2) presented below, while using the value of the image feature value or the value of the mMAF as the feature value.

$$( 1 )$$

$$STANDARDIZED FEATURE VALUE = \frac{\begin{matrix}ORIGINAL FEATURE VALUE - \\ AVERAGE OF ORIGINAL FEATURE VALUES \\ IN INPUT DATA GROUP\end{matrix}}{\begin{matrix}STANDARD DEVIATION OF ORIGINAL FEATURE \\ VALUES IN INPUT DATA GROUP\end{matrix}}$$

$$( 2 )$$

$$NORMALIZED FEATURE VALUE = \frac{\begin{matrix}ORIGINAL FEATURE VALUE - \\ MINIMUM VALUE AMONG ORIGINAL FEATURE \\ VALUES IN INPUT DATA GROUP\end{matrix}}{\begin{matrix}MAXIMUM VALUE AMONG ORIGINAL FEATURE \\ VALUES IN INPUT DATA GROUP - \\ MINIMUM VALUE AMONG ORIGINAL FEATURE \\ VALUES IN INPUT DATA GROUP\end{matrix}}$$

For example, at the time of performing the process of specifying the genes that may exhibit a false negative, the specifying function 354 is configured to calculate the numerical values presented in FIG. 5, by performing the standardization process presented in Expression (1) or the normalization process presented in Expression (2) above on the mMAF (or the MAF). Alternatively, the calculating function 355 may calculate the tissue specimen mutation scores based on the MAF, by performing the standardization process presented in Expression (1) or the normalization process presented in Expression (2) above on the mMAF (or the MAF). Further, the calculating function 355 is configured to calculate the numerical values presented in FIG. 5, by performing the standardization process presented in Expression (1) or the normalization process presented in Expression (2) above on the values of the image feature values calculated from the medical image.

The calculating function 355 is configured to store, in the storage circuitry 34, the calculated tissue specimen mutation scores so as to be kept in correspondence with the subject information.

### The Tissue Specimen Mutation Score Display Process

As explained at step S108 in FIG. 4, the controlling function 351 is configured to cause the display 33 to display the tissue specimen mutation scores calculated by the calculating function 355. In this situation, the controlling function 351 is capable of exercising control so that the tissue specimen mutation scores based on the medical image and the tissue specimen mutation scores based on the data related to the ctDNA are displayed in a distinguishable manner.

FIG. 7 is a drawing illustrating an example of display information according to the first embodiment. For example, as illustrated in FIG. 7, at the time of displaying the tissue specimen mutation scores of gene groups A to E, the controlling function 351 is configured to use mutually-different display modes (e.g., to display the backgrounds in mutually-different colors) for the values based on the medical image (the image feature values) and for the values based on the ctDNA and is thus able to realize the display indicating, in a distinguishable manner, which base was used for calculating the value.

### The Treatment Effect Judging Process

As explained at steps S109 and S110 in FIG. 4, when it is determined that the treatment effect is to be judged, the judging function 356 is configured to judge the effect of the treatment for the cancer patient. More specifically, when an operation to carry out the treatment effect judging process is performed via the input interface 32 or when the subject is a subject for whom a tissue specimen mutation score was calculated in the past, the judging function 356 determines that the treatment effect is to be judged at step S109.

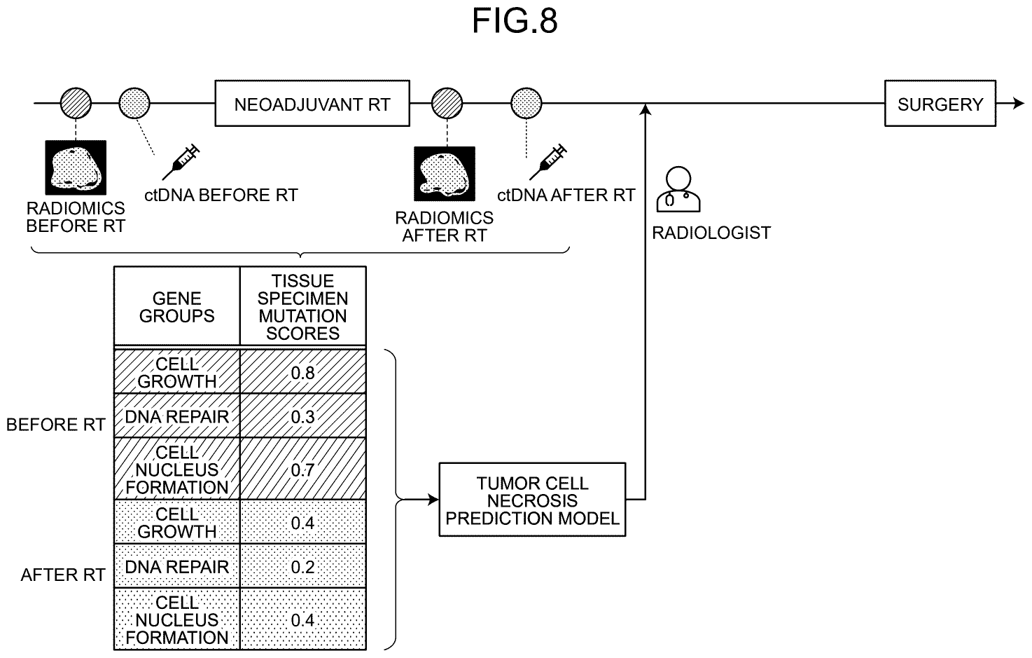

After that, the judging function 356 is configured to read the previous tissue specimen mutation scores for the relevant subject from the storage circuitry 34 and to judge the treatment effect by using the read previous tissue specimen mutation scores and the tissue specimen mutation scores calculated at step S108. FIG. 8 is a drawing for explaining an example of the process performed by the judging function 356 according to the first embodiment. In this situation, FIG. 8 illustrates an example in which, on the basis of tissue specimen mutation scores calculated before and after radiation treatment (“neoadjuvant RT”) performed prior to surgery, an effect of the radiation treatment prior to the surgery is judged.

In this situation, for example, as illustrated in FIG. 8, a medical image is acquired and a liquid biopsy test is performed, before the radiation treatment (“before RT”) prior to the surgery so that, on the basis of these results, a pre-radiation-treatment tissue specimen mutation score is calculated with respect to each of the gene groups (cell growth, DNA repair, and cell nucleus formation). In addition, a medical image is acquired and a liquid biopsy test is performed, after the radiation treatment (“after RT”) prior to the surgery so that, on the basis of these results, a post-radiation-treatment tissue specimen mutation score is calculated with respect to each of the gene groups (cell growth, DNA repair, and cell nucleus formation).

The judging function 356 is configured to obtain information indicating the treatment effect, by inputting the pre-radiation-treatment tissue specimen mutation scores corresponding to the gene groups and the post-radiation-treatment tissue specimen mutation scores corresponding to the gene groups to a tumor cell necrosis prediction model. For example, the tumor cell necrosis prediction model is generated in advance through machine learning which uses, as training data, tissue specimen mutation scores before and after treatment and information indicating treatment effects (e.g., numerical values assigned to treatment effects in actuality), so that the model is stored into the storage circuitry 34.

When the judging function 356 has obtained the information indicating the treatment effect, the controlling function 351 is configured to cause the display 33 to display a result of the judgment (the information indicating the treatment effect). A multidisciplinary clinical team including radiation oncologist, surgeon, and radiologist makes a judgment about treatment in the future (additional radiation treatment, surgery, etc.) on the basis of the judgment result displayed on the display 33.

Although the example was explained with reference to FIG. 8 in which the treatment effect is judged by using the tumor cell necrosis prediction model, possible embodiments are not limited to this example. It is also acceptable to judge the treatment effect by using other methods. For example, it is acceptable to judge the treatment effect on the basis of increases and decreases of the tissue specimen mutation scores.

As explained above, according to the first embodiment, the obtaining function 352 is configured to obtain the data related to the ctDNA of the subject. The specifying function 354 is configured to specify the genes, on the basis of the information about false negatives in the results of estimating the genetic mutations in the tumor tissue of the subject based on the data related to the ctDNA. With respect to the genes specified on the basis of the information related to false negatives, the calculating function 355 is configured to calculate the results of estimating the genetic mutations in the tumor tissue, on the basis of the medical image of the tumor tissue of the subject. Consequently, with respect to the genes having the possibility of exhibiting a false negative, the medical information processing apparatus 3 according to the first embodiment is able to estimate the genetic mutations on the basis of the medical image and thus makes it possible to enhance the level of precision for estimating the genetic mutations in the tumor tissue.

Further, according to the first embodiment, the specifying function 354 is configured to specify the first gene having a high possibility of exhibiting a false negative in the results of estimating the genetic mutations based on the data related to the ctDNA. With respect to the first gene, the calculating function 355 is configured to calculate the tissue specimen mutation score for estimating the genetic mutations on the basis of the medical image of the tumor tissue. With respect to the second gene other than the first gene, the calculating function 355 is configured to calculate the tissue specimen mutation score for estimating the genetic mutations on the basis of the data related to the ctDNA. Consequently, the medical information processing apparatus 3 according to the first embodiment is capable of estimating the genetic mutations in the tumor tissue by combining the ctDNA data with the medical image and thus makes it possible to enhance capabilities for estimating the genetic mutations in the tumor tissue.

Further, according to the first embodiment, the specifying function 354 is configured to judge the possibility of exhibiting a false negative in the results of estimating the genetic mutations, on the basis of the MAF of each of the genes. Further, the specifying function 354 is configured to specify each of the genes of which the MAF value is smaller than the threshold value as the first gene having a high possibility of exhibiting a false negative. Consequently, the medical information processing apparatus 3 according to the first embodiment makes it possible to easily judge the possibility of exhibiting a false negative.

Further, according to the first embodiment, the calculating function 355 is configured to calculate the image feature values in the medical image of the tumor tissue, as the result of estimating the mutations in the gene specified on the basis of the information about false negatives. Consequently, the medical information processing apparatus 3 according to the first embodiment makes it possible to estimate the genetic mutations with a constant level of precision.

Further, according to the first embodiment, the controlling function 351 is configured to exercise control so that the tissue specimen mutation scores based on the medical image and the tissue specimen mutation scores based on the data related to the ctDNA are displayed in the distinguishable manner. Consequently, the medical information processing apparatus 3 according to the first embodiment makes it possible to provide the user with the information that was used at the time of calculating the tissue specimen mutation scores.

In addition, according to the first embodiment, the judging function 356 is configured to judge the treatment effect on the tumor tissue, on the basis of the results of estimating the genetic mutations in the tumor tissue before and after the treatment performed on the tumor tissue of the subject. Consequently, the medical information processing apparatus 3 according to the first embodiment makes it possible to provide the user with the treatment effect.

Also, according to the first embodiment, the classifying function 353 is configured to classify the genes into the gene groups. The specifying function 354 is configured to specify the gene groups each having a high possibility of exhibiting a false negative in the estimation result regarding the genetic mutations estimated with respect to each of the gene groups. With respect to the specified gene groups, the calculating function 355 is configured to calculate the result of estimating the genetic mutations in the tumor tissue, on the basis of the medical image of the tumor tissue of the subject. Consequently, the medical information processing apparatus 3 according to the first embodiment makes it possible to address even the situation where it is difficult to perform a significance test on the image feature values. There are a large number of types of genetic mutations in relation to the number of subjects, and there are some situations where performing a significance test on image feature values would be difficult if gene mutations of a single gene were used. To cope with those situations, by forming the gene groups as described above, performing the significance test on the image feature values is made possible.

Further, according to the first embodiment, the classifying function 353 is configured to classify the genes into the gene groups in accordance with the functions of the genes. Consequently, the medical information processing apparatus 3 according to the first embodiment makes it possible to organize the certain genes that are useful in judging the treatment effect into the groups.

### Other Embodiments

In the above embodiments, the example was explained in which the gene groups are formed so as to calculate the tissue specimen mutation score with respect to each of the gene groups; however, possible embodiments are not limited to this example. It is also acceptable, as explained with reference to FIG. 3, to calculate a tissue specimen mutation score with respect to each of the single genes.

The processing circuitry explained in the above embodiments may be structured by combining together a plurality of independent processors, so that the processing functions are realized as a result of the processors executing the programs. Further, the processing functions of the processing circuitry may be realized as being distributed among or integrated into one or more pieces of processing circuitry, as appropriate. Furthermore, the processing functions of the processing circuitry may be realized by using a combination of software and hardware such as circuitry. Further, although the example was explained in which the programs corresponding to the processing functions are stored in the single piece of storage circuitry (the storage circuitry 34), possible embodiments are not limited to this example. For instance, another configuration is also acceptable in which the programs corresponding to the processing functions are stored in a plurality of pieces of storage circuitry in a distributed manner, so that the processing circuitry reads and executes the programs from the pieces of storage circuitry.

Further, in the above embodiments, the example was explained in which the functional units in the present disclosure are realized as the functions of the processing circuitry, respectively; however, possible embodiments are not limited to this example. For instance, instead of being realized as the functions described in the embodiments, the functions of the functional units of the present disclosure may be realized by hardware alone, software alone, or a mixture of hardware and software.

Further, the term “processor” used in the description of the above embodiments may denote, for example, a Central Processing Unit (CPU), a Graphics Processing Unit (GPU), or circuitry such as an Application Specific Integrated Circuit (ASIC) or a programmable logic device (e.g., a Simple Programmable Logic Device (SPLD), a Complex Programmable Logic Device (CPLD), or a Field Programmable Gate Array (FPGA)). In this situation, instead of having the programs saved in the storage circuitry, it is also acceptable to directly incorporate the programs into the circuitry of one or more processors. In that situation, the one or more processors are configured to realize the functions by reading and executing the programs incorporated in the circuitry thereof. Further, the processors of the present embodiments do not each necessarily need to be structured as a single piece of circuitry. It is also acceptable to structure one processor by combining together a plurality of pieces of independent circuitry so as to realize the functions thereof.

In relation to the above, a medical information processing program executed by the one or more processors is provided as being incorporated, in advance, in a Read-Only Memory (ROM), storage circuitry, or the like. Further, the medical information processing program may be provided as being recorded on a non-transitory computer-readable storage medium such as a Compact Disk Read-Only memory (CD-ROM), a Floppy Disk (FD), a Compact Disk Recordable (CD-R), or a Digital Versatile Disk (DVD), in a file in a format that is installable or executable by those apparatuses. Further, the medical information processing program may be stored in a computer connected to a network such as the Internet so as to be provided or distributed as being downloaded via the network. For example, the medical information processing program is structured with modules including the processing functions described above. In the actual hardware, as a result of a CPU reading and executing the medical information processing program from a storage medium such as a ROM, the modules are loaded into a main storage apparatus and generated in the main storage apparatus.

In the embodiments and the modification examples described above, the constituent elements of the apparatuses illustrated in the drawings are based on functional concepts. Thus, it is not necessarily required to physically configure the constituent elements as indicated in the drawings. In other words, specific modes of distribution and integration of the apparatuses are not limited to those illustrated in the drawings. It is acceptable to functionally or physically distribute or integrate all or a part of the apparatuses in any arbitrary units, depending on various loads and the status of use. Further, all or an arbitrary part of the processing functions performed by the apparatuses may be realized by a CPU and a program analyzed and executed by the CPU or may be realized as hardware using wired logic.

Furthermore, with regard to the processes explained in the embodiments and the modification examples described above, it is acceptable to manually perform all or a part of the processes described as being performed automatically. Conversely, by using a publicly-known method, it is also acceptable to automatically perform all or a part of the processes described as being performed manually. Further, unless noted otherwise, it is acceptable to arbitrarily modify any of the processing procedures, the controlling procedures, specific names, and various information including various types of data and parameters that are presented in the above text and the drawings.

According to at least one aspect of the embodiments described above, it is possible to enhance the level of precision for estimating the genetic mutations in the tumor tissue.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry configured to
obtain data related to circulating tumor deoxyribonucleic acid (ctDNA) of a subject;
specify a gene on a basis of information about a false negative in a result of estimating a genetic mutation in a tumor tissue of the subject based on the data related to the ctDNA; and
calculate, with respect to the gene specified on the basis of the information about the false negative, a result of estimating the genetic mutation in the tumor tissue, on a basis of a medical image of the tumor tissue of the subject.

2. The medical information processing apparatus according to claim 1, wherein
the processing circuitry is configured to specify a first gene having a high possibility of exhibiting a false negative in the result of estimating the genetic mutation based on the data related to the ctDNA, and
with respect to the first gene, the processing circuitry is configured to calculate a mutation score for estimating the genetic mutation on a basis of the medical image of the tumor tissue, whereas
with respect to a second gene other than the first gene, the processing circuitry is configured to calculate a mutation score for estimating the genetic mutation on a basis of the data related to the ctDNA.

3. The medical information processing apparatus according to claim 2, wherein the processing circuitry is configured to judge the possibility of exhibiting the false negative in the result of estimating the genetic mutation, on a basis of mutant allele frequency (MAF) of each gene.

4. The medical information processing apparatus according to claim 3, wherein the processing circuitry is configured to specify a gene having an MAF value smaller than a threshold value as the first gene having the high possibility of exhibiting the false negative.

5. The medical information processing apparatus according to claim 1, wherein the processing circuitry is configured to calculate an image feature value in the medical image of the tumor tissue, as a result of estimating a mutation in the gene specified on the basis of the information about the false negative.

6. The medical information processing apparatus according to claim 2, wherein the processing circuitry is configured to exercise control so that the mutation score based on the medical image and the mutation score based on the data related to the ctDNA are displayed in a distinguishable manner.

7. The medical information processing apparatus according to claim 1, wherein, on the basis of results of estimating the genetic mutation in the tumor tissue before and after treatment performed on the tumor tissue of the subject, the processing circuitry is configured to judge an effect of the treatment on the tumor tissue.

8. The medical information processing apparatus according to claim 1, wherein
the processing circuitry is further configured to classify genes into gene groups,
the processing circuitry is configured to specify a gene group having a high possibility of exhibiting a false negative in an estimation result regarding the genetic mutation estimated with respect to each of the gene groups, and
with respect to the specified gene group, the processing circuitry is configured to calculate the result of estimating the genetic mutation in the tumor tissue, on the basis of the medical image of the tumor tissue of the subject.

9. The medical information processing apparatus according to claim 8, wherein the processing circuitry is configured to classify the genes into the gene groups in accordance with functions of the genes.

10. A storage medium storing therein, in a non-transitory manner, a program that causes a computer to execute:
obtaining data related to circulating tumor deoxyribonucleic acid (ctDNA) of a subject;
specifying a gene on a basis of information about a false negative in a result of estimating a genetic mutation in a tumor tissue of the subject based on the data related to the ctDNA; and
calculating, with respect to the gene specified on the basis of the information about the false negative, a result of estimating the genetic mutation in the tumor tissue, on a basis of a medical image of the tumor tissue of the subject.

11. A method comprising:
obtaining data related to circulating tumor deoxyribonucleic acid (ctDNA) of a subject;
specifying a gene on a basis of information about a false negative in a result of estimating a genetic mutation in a tumor tissue of the subject based on the data related to the ctDNA; and
calculating, with respect to the gene specified on the basis of the information about the false negative, a result of estimating the genetic mutation in the tumor tissue, on a basis of a medical image of the tumor tissue of the subject.
